(19) United States
(12) Patent Application Publication
(10) Pub. No.: US 2026/0310978 A1
(43) Pub. Date: Oct. 8, 2026

(54) SYSTEM AND METHOD FOR DETECTING PATHOGENS USING ARTIFICIAL INTELLIGENCE

(71) Applicant: TECHNOLOGIE BIOEUREKA INC., Montréal (CA)

(72) Inventor: Mahamadou Mohamed DIALLO, Lévis (CA)

(21) Appl. No.: 19/482,074

(22) PCT Filed: May 9, 2024
(86) PCT No.: PCT/CA2024/050629

§ 371 Date: Nov. 6, 2025

Related U.S. Application Data
(60) Provisional application No. 63/501,004, filed on May 9, 2023.

(51) Int. Cl. G06N 3/09; G06N 3/092; G16H 70/60

(52) U.S. Cl. CPC G06N 3/09; G06N 3/092; G16H 70/60

(57) ABSTRACT

Method, system, device, and computer program, for detecting pathogens in samples. One or more micrographs of the sample is captured; features are extracted from the micrographs; a trained pathogen model recognizes the pathogens in the sample; a report is provided back to the user. Micrographs may be submitted to a training dataset. Manual review may be requested. Method, system, device, and computer program, for training a pathogen model. capture one or more historical micrographs of a historical sample. The pathogen model is trained on a pathogen dataset and validated using a testing subset of the pathogen dataset.

### TECHNICAL FIELD

The present invention relates to microbiology and, more particularly, to pathogen identification from samples.

### BACKGROUND

Even though some more sophisticated methods may exist for pathogen identification, microscopy remains a complementary, if not the primary method, in many scenarios. It is often regarded as a rapid, cost-effective and field-friendly method thanks to the portability of the required equipment. The volume of samples being analyzed can, however, be limited by the availability of trained microbiologist with the required knowledge of microbial morphology. The present disclosure aims at alleviating this obstacle.

### SUMMARY

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In a first aspect, the technique described herein relates to devices, systems, computer programs and methods for identifying pathogens from a sample.

In a second aspect, the technique described herein relates to devices, systems, computer programs and methods for training a pathogen model.

In accordance with the first aspect, a method for identifying pathogens from a sample is provided. The method includes capturing one or more micrographs of the sample, recognizing one or more pathogens from the one or more micrographs using a trained pathogen model, and preparing a pathogen report comprising one or more recognized pathogen labels.

In accordance with the first aspect, a system for identifying pathogens from a sample is provided. The system includes a capture module, a classifier module, one or more processors, and a user interface module. The capture module may capture one or more micrographs of the sample. The classifier module may recognize one or more pathogens from the one or more micrographs using a trained pathogen model. The one or more processors may prepare a pathogen report comprising one or more recognized pathogen labels. The user interface module may present the pathogen report. Optionally, a microscope may adjust a focus, a magnification and a sample position until the one or more pathogens are visible on a video stream. Optionally, a network interface module may request a manual identification of the one or more micrographs and receive the manual identification of the one or more micrographs.

In accordance with the first aspect, a device for identifying pathogens from a sample is provided. The device may include a capture module configured to capture one or more micrographs of the sample, a classifier module is configured to recognize one or more pathogens from the one or more micrographs using a trained pathogen model, one or more processors configured to prepare a pathogen report comprising one or more recognized pathogen labels, and a user interface module configured to present the pathogen report.

In accordance with the first aspect, a non-transitory computer-readable medium storing a set of instructions for identifying pathogens from a sample is provided. The non-transitory computer-readable medium storing a set of instructions may include one or more instructions that, when executed by one or more processors of a device, cause the device to capture one or more micrographs of the sample, recognize one or more pathogens from the one or more micrographs using a trained pathogen model, and prepare a pathogen report comprising one or more recognized pathogen labels.

In embodiments, the one or more micrographs and the one or more recognized pathogen labels may be contributed to a training dataset.

In embodiments, a video stream may be received from a microscope. A focus of the microscope, a magnification of the microscope and a sample position may be adjusted until the one or more pathogens are visible on the video stream. The one or more micrographs may be captured from one or more images of the video stream.

In embodiments, scientific information about the one or more recognized pathogen labels may be searched within a scientific information repository. The scientific information about the one or more recognized pathogen labels may be added to the pathogen report.

In embodiments, to recognize the one of more pathogens, one or more features may be extracted from the one or more micrographs and the one or more pathogens may be recognized from the one or more features.

In embodiments, the one or more features may be extracted using a convolutional neural network (CNN) trained to detect the one or more pathogens.

In embodiments, the one or more features may be extracted using the trained pathogen model, the trained pathogen model comprising a convolutional neural network (CNN) trained to detect the one or more pathogens.

In embodiments, to extract the one or more features, a plurality of pathogen boundaries may be iteratively located within the one or more micrographs and the one or more features may be extracted from the plurality of pathogen boundaries. A proportion of the one or more recognized pathogen labels with respect to plurality of pathogen boundaries may be computed and added to the pathogen report.

In embodiments, a confidence score may be assigned to the pathogen boundaries. The plurality of pathogen boundaries may be iteratively located until the confidence score of a best pathogen boundary candidate is below a pathogen boundary confidence threshold.

In embodiments, a manual identification may be requested of the one or more micrographs. A manual pathogen report may be prepared with one or more manual pathogen labels upon completing the manual identification.

In embodiments, the one or more recognized pathogen labels may include at least one of a Salmonella spp, a Listeria monocytogene, an Escherichia coli(E. coli), a Campylobacter jejuni, a Staphylococcus aureus, a Mycobacterium bovis, a Cryptosporidium, a Toxoplasma gondii, an Aspergillus spp, a Penicillium spp, a Fusarium spp, a Botrytis cinerea, and a Pseudomonas aeruginosa.

In embodiments, the trained pathogen model may include at least one of a residual network (ResNet) deep learning model, a few-shot learning (FSL) model, and a combination thereof.

In embodiments, an identification confidence for each of the one or more recognized pathogen labels may be computed and added to the pathogen report.

In embodiments, the trained pathogen model may be acquired while being connected to a remote pathogen model repository and the one or more pathogens may be recognized while being disconnected from the remote pathogen model repository.

In embodiments, the one or more micrographs and the one or more recognized pathogen labels may be enqueued for one or more micrographs contribution while being disconnected from a remote training dataset repository and the one or more micrographs contribution to a training dataset may be contributed upon reconnecting to the remote training dataset repository.

In embodiments, the classifier module may be one of a local classifier module, a remote classifier module and a combination thereof.

In embodiments, the one or more recognized pathogen labels may include at least one of a family, a name, a strain, a serotype, a genetic information, and a virulence factor.

In embodiments, the one or more recognized pathogen labels include one or more pathogen family labels.

In accordance with the second aspect, a method for training a pathogen model is provided. The method includes capturing one or more historical micrographs of a historical sample, applying a historical pathogen label on the one or more historical micrographs into one or more labeled historical micrographs and contributing the one or more labeled historical micrographs to a pathogen dataset. The method further includes training the pathogen model using supervised reinforcement learning on a training subset of the pathogen dataset and validating the pathogen model using a testing subset of the pathogen dataset.

In accordance with the second aspect, a system for training a pathogen model is provided. The system includes one or more processors. The one or more processors may capture one or more historical micrographs of a historical sample, apply a historical pathogen label on the one or more historical micrographs into one or more labeled historical micrographs and contribute the one or more labeled historical micrographs to a pathogen dataset. The one or more processors may further train the pathogen model using supervised reinforcement learning on a training subset of the pathogen dataset and validate the pathogen model using a testing subset of the pathogen dataset.

In accordance with the second aspect, a device for training a pathogen model is provided. The device may include one or more processors configured to capture one or more historical micrographs of a historical sample, apply a historical pathogen label on the one or more historical micrographs into one or more labeled historical micrographs, contribute the one or more labeled historical micrographs to a pathogen dataset, train the pathogen model using supervised reinforcement learning on a training subset of the pathogen dataset, and validate the pathogen model using a testing subset of the pathogen dataset.

In accordance with the second aspect, a non-transitory computer-readable medium storing a set of instructions for training a pathogen model is provided. The non-transitory computer-readable medium storing a set of instructions for may include one or more instructions that, when executed by one or more processors of a device, cause the device to capture one or more historical micrographs of a historical sample, apply a historical pathogen label on the one or more historical micrographs into one or more labeled historical micrographs, contribute the one or more labeled historical micrographs to a pathogen dataset, train the pathogen model using supervised reinforcement learning on a training subset of the pathogen dataset; and validate the pathogen model using a testing subset of the pathogen dataset.

In embodiments, the one or more labeled historical micrographs may be transformed into one or more altered historical micrographs and the one or more altered historical micrographs may be contributed to a training dataset. Optionally, the one or more labeled historical micrographs may be transformed using at least one of a blurring, a scaling, a rotation, a skewing, and a noise addition.

In embodiments, one or more new micrographs may be acquired of a new sample, the one or more new micrographs may be labeled into one or more labeled new micrographs. The pathogen model may be finetuned using the one or more labeled new micrographs into a finetuned pathogen model.

### BRIEF DESCRIPTION OF THE DRAWINGS

Further features and exemplary advantages of the present invention will become apparent from the following detailed description, taken in conjunction with the appended drawings, in which:

FIG. 1 is a flow chart depicting an exemplary method embodiment for identifying pathogens from a sample in accordance with the teachings of the present invention;

FIG. 2 is a flow chart depicting an exemplary method embodiment for identifying pathogens from a sample in accordance with the teachings of the present invention;

FIG. 3 is a flow chart depicting an exemplary method embodiment for identifying pathogens from a sample in accordance with the teachings of the present invention;

FIG. 4 is a flow chart depicting an exemplary method embodiment for training a pathogen model, in accordance with the teachings of the present invention;

FIG. 5 is a logical modular representation of an exemplary system for identifying pathogens from a sample in accordance with the teachings of the present invention.

### DETAILED DESCRIPTION

The present description is made with reference to the accompanying drawings, in which example embodiments are shown. However, many different embodiments may be used, and thus the description should not be construed as limited to the particular embodiments set forth herein. Like numbers refer to like elements throughout.

As will be appreciated by one of skill in the art upon reading the following disclosure, various aspects described herein may be embodied as a device, a method or a computer program product (e.g., a non-transitory computer-readable medium having computer executable instruction for performing the noted operations or steps). Accordingly, those aspects may take the form of an entirely hardware embodiment, an entirely software embodiment, or an embodiment combining software and hardware aspects.

Furthermore, such aspects may take the form of a computer program product stored by one or more computer-readable storage media having computer-readable program code, or instructions, embodied in or on the storage media. Any suitable computer readable storage media may be utilized, including hard disks, CD-ROMs, optical storage devices, magnetic storage devices, and/or any combination thereof.

The embodiments described herein may be useful for healthcare providers to obtain pathogen analysis even when a trained microbiologist is unavailable. The methods, systems, devices, and computer programs may help reduce delay in obtaining the pathogen content and ultimately result in more effective treatments for patients. The methods, systems, devices, and computer programs may also be useful for other professionals such as public health agencies, research institutions, food and agriculture industries and environmental industries which may also benefit from quicker pathogen analysis. The embodiments described herein may also be useful for other professionals such as public health agencies, research institutions, food and agriculture industries and environmental industries which may also benefit from quicker pathogen analysis.

A first aspect of the teachings presented herein relates to devices, systems, computer programs and methods for identifying pathogens from a sample that, in a first set of embodiments, can be used to obtain a report of pathogen content within a sample.

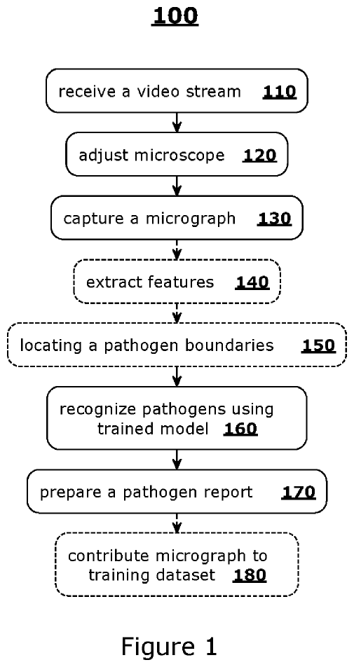

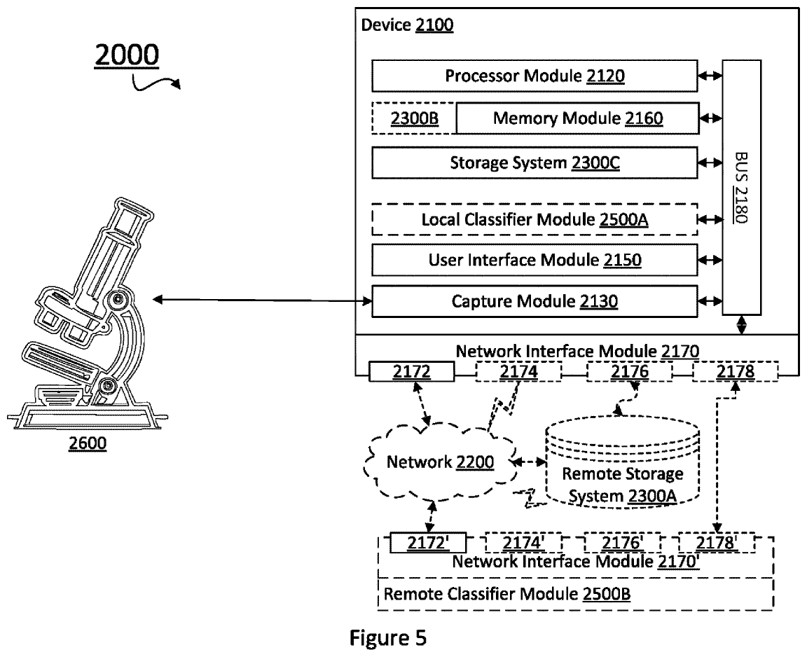

Reference is now made to the drawings in which FIG. 1 shows a flow chart of an exemplary method embodiment 100 for identifying pathogens from a sample and FIG. 5 shows a logical modular representation of an exemplary system 2000 comprising a pathogen identifying device 2100 in accordance with the teachings of the present invention.

In certain embodiments, the method 100 includes capturing 130 one or more micrographs of the sample, recognizing 160 one or more pathogens from the one or more micrographs using a trained pathogen model, and preparing 170 a pathogen report comprising one or more recognized pathogen labels. When embodied as a system or device, a capture module 2130 may capture 130 one or more micrographs of the sample, a classifier module 2500 may recognize 160 one or more pathogens from the one or more micrographs using a trained pathogen model, and one or more processors 2120 may prepare 170 a pathogen report comprising one or more recognized pathogen labels. The user interface module 2150 may present the pathogen report.

A micrograph may refer to a photograph or an image that is taken of a sample using a microscope. Micrographs may be a visual representation of the microscopic details and structures of the specimen under examination.

Samples may include, for example, biological fluids, tissue samples, environmental samples, animal samples, and food and beverage samples. The samples may contain pathogens such as parasites, fungi, viruses and bacteria.

The pathogen labels may include the name of the pathogen and additional information such as the family, the strain, the serotype, genetic information, the virulence factors, and the method of identification.

For example, the one or more recognized pathogen labels may include at least one of a Salmonella spp, a Listeria monocytogene, an Escherichia coli(E. coli), a Campylobacter jejuni, a Staphylococcus aureus, a Mycobacterium bovis, a Cryptosporidium, a Toxoplasma gondii, an Aspergillus spp, a Penicillium spp, a Fusarium spp, a Botrytis cinerea, and a Pseudomonas aeruginosa.

To capture 130 the micrographs using a regular microscope 2600, a microscope attachment device may be installed onto ocular lenses. The microscope attachment device may comprise a capture sensor and a communication module from which the captured images may be received 110 as a video stream to the pathogen identifying device 2100. The communication may be achieved through a wired communication (e.g., USB, Ethernet, HDMI) or a wireless communication (e.g., Wifi, Bluetooth). In other embodiments, the microscope 2600 may be equipped with its own internal capture sensor and communication module from which the video stream may be received 110 without the microscope attachment device. In another embodiment, the pathogen identifying device 2100 may be embedded within the microscope 2600 such that the video stream is received 110 from a communication bus.

In embodiments, a video stream may be received 110 from a microscope. A focus of the microscope, a magnification of the microscope and a sample position may be adjusted 120 until the one or more pathogens are visible on the video stream. The micrographs may be captured 130 from one or more images of the video stream.

Capturing the micrographs, in the context of this disclosure may include sampling and digitizing an image as viewable by the optical system of the microscope. Although other sampling rates may be considered, the sampling frequency may be between 15 Hz and 60 Hz such that motion of the pathogen may be accurately captured and adjustment to the microscope (e.g., adjustment in focus and magnification) may be displayed in a responsive manner to a user (e.g., within 250 ms). The digitized frames may be captured with a precision of 8 bits per component, although some embodiments may benefit from higher precision such as 10 bits, 12 bits, 16 bits or even higher. In embodiments, red, green and blue components may be captured. In embodiments, frames may be captured in grayscale. In embodiments, other color spaces may be used, including luminosity-chroma spaces such as YCbCr such that more precision may be used to represent the luminosity than hue and saturation.

In embodiments, the capture sensor may be configured to capture wavelengths not visible with the naked eye. For example, the capture sensor may be capable of capture UV and/or infrared light.

In embodiments, the capture sensor may be configured to more than one view from the microscope, such as a binocular pair. The binocular pair may be used to generate a 3D model of the captured sample, including depth. The multiple images may be captured by different sensors as different independent video streams, or captured as a single stream, where the multiple views are captured together (e.g., in a left-right or top-bottom configuration), or captured as a single video stream with alternating frames corresponding to alternating views (e.g., odd/even), or as different channels within the same video stream. When captured by different sensors, the sensors may be synchronized using a generator locking signal. Depth and 3D models may be obtained from the multiple views through stereovision algorithms. In multi-view or even single-view configurations, depth from focus/defocus algorithms may also be used to extract depth, where the depth of the sample may be estimated by analyzing the variations in focus or blur across a series of images taken with different focus settings.

When external to the microscope 2600, the pathogen identifying device 2100 may be embodied by a regular smart phone device, a tablet, a laptop or a regular desktop or workstation computer.

The one or more processors 2120 may comprise CPUs, FPGAs, and GPUS capable of executing the instructions of a computer program. The classifier module 2500 may broadly refer to a subset of the pathogen identifying device 2100 specialized for the identification of pathogens from micrographs, comprising computer instructions, ephemeral memory storage such as RAM used for processing the instructions and data, persistent memory storage (local or remote) used to load and save a large amount of data, including various models such as the trained pathogen model. The user interface module 2150 may comprise a display device used to provide visual feedback to a user, including reports and interactive widgets. The user interface module 2150 may further comprise interacting devices such as a keyboard, a mouse, a trackpad, a microphone, or a touchscreen device.

The microscope 2600 may be capable of adjusting 120 a focus, a magnification and a sample position until the one or more pathogens are visible on a video stream. The microscope adjustments may be achieved manually by a user, or electronically from the user interface module 2150. The microscope adjustments may position the sample such that a region of interest of the sample being captured by the capture sensor. The microscope magnification may be adjusted to such that the pathogen may be fully visible by the capture sensor and yet at a resolution such that the shape and internal features of the pathogen are visible. The focus may be adjusted to ensure that the shape and internal features of the pathogen are sharp. In embodiments, depth of field bracketing may be used, where the focus may be transitioning from the upper region to the lower region (or vice-versa) of the pathogen in order to capture the entire volume of the pathogen or capture depth information. In embodiment, the positioning, focusing and magnification may be assisted such at, after an approximate configuration, the microscope configuration further adjusted automatically. For example, automatic adjustments may include position adjustments to ensure that the sample is centered. Depth of field bracketing may also be automated. Magnification bracketing may also be performed such that different resolutions of the pathogens are captured. Exposure bracketing may also be used to achieve HDR microscopy, either by changing the capture sensor shutter speed, or by adjusting the microscope illumination. Other types of adjustments may include adjusting a polarization filter, or capturing using polarization bracketing, adjusting the illumination frequency for fluorescence microscopy, or capturing using frequency bracketing, adjusting the angle of illumination, or capturing using angle bracketing.

The pathogens captured by a micrograph may be recognized 160, or identified, using a trained pathogen model.

Before being recognized 160, the one of more pathogens, one or more features may be extracted 140 from the micrograph and the one or more pathogens may be recognized from the one or more features. Before feature extraction, the micrograph may undergo preprocessing to enhance the image quality, including noise reduction, contrast adjustment, and normalization to ensure that the features extracted 140 are representative of the pathogens rather than artifacts of the image. Extracted features may include predefined features based on domain knowledge, such as shape, size, texture, color intensity, and morphological characteristics specific to pathogens.

In embodiments, the one or more features may be extracted 140 using a convolutional neural network (CNN) trained to detect the one or more pathogens. The features may also include automatically learned features from the data using techniques such as Convolutional Neural Networks (CNNs). The extracted features may be transformed into a numerical format (vector representation) that can be processed by machine learning models. This representation captures the essential information needed for distinguishing between different pathogens. Contrast stretching and histogram equalization may be used to improve the visibility of features within a micrograph by adjusting the image's contrast. Contrast stretching many increase the dynamic range of the levels in the image, making the underlying structures more distinguishable, while histogram equalization may redistribute the micrograph's intensity distribution, enhancing contrast and revealing hidden features in areas that were previously too bright or too dark. Skeletonization, such as medial axis transform, may be used to reduce shapes in the image to their skeletal form, preserving the structure's overall geometry while minimizing complexity. A Medial Axis Transform may provide a “skeleton” by identifying the set of points equidistant from the shape's boundaries enabling, for example, analysis of the structural properties of pathogens, such as their elongation or branching patterns. Shape context extraction may be used to capture the shape of an object by considering the distribution of contour points relative to each other and may highlight unique morphological features of some pathogens. Fourier descriptors may be used to simplify complex shapes by transforming their outlines into a series of coefficients derived from the Fourier Transform. Fourier descriptors may be useful for capturing the periodicity and symmetry of pathogen shapes, allowing for the recognition of pathogens based on their outline's frequency components. Curvature Scale Space (CSS) may be used to represent the curvature of contours at multiple scales, providing a multi-scale representation of an object's shape. CSS may capture shape features that are invariant to rotation, translation, and scale.

In embodiments, the one or more features may be extracted 140 using the trained pathogen model, the trained pathogen model comprising a convolutional neural network (CNN) trained to detect the one or more pathogens. The trained pathogen, further described hereinbelow may, for example, already include a trained CNNs or feature extraction through ResNet models.

In embodiments, to extract 140 the one or more features, a plurality of pathogen boundaries may be iteratively located 150 within the micrograph and the one or more features may be extracted 140 from the plurality of pathogen boundaries. A proportion of the one or more recognized pathogen labels with respect to plurality of pathogen boundaries may be computed and added to the pathogen report. Locating 150 boundaries may be useful in situations where the micrographs contain a wide view of the sample with potentially multiple pathogens. The location of individual pathogens may be located 150 such that the identification 160 may then be performed on individual pathogens. When multiple pathogens are located within a micrograph, the pathogen identification 160 may be performed on a single pathogen, or on each pathogen, individually. When performed on each pathogen individually, a proportion of each type of pathogen identified 160 within the micrograph may be computed and reported to the user.

In embodiments, a confidence score may be assigned to the pathogen boundaries. The plurality of pathogen boundaries may be iteratively located 150 until the confidence score of a best pathogen boundary candidate is below a pathogen boundary confidence threshold. The score may, for example, be lower when multiple pathogens on the micrograph overlap, or when the pathogen on the micrograph is partially occluded or partially outside of the micrograph.

Features of the micrograph may be compared against patterns the model has learned to associate with specific pathogens during its training phase. Each label in the training dataset may indicate the presence or absence of particular pathogens, allowing the model to learn the unique characteristics-such as shape, size, texture, and color-that distinguish each pathogen. When a new micrograph is presented, the model may analyze the image to detect these learned features and applies the knowledge it has acquired to predict the likelihood of each known pathogen's presence in the sample.

In embodiments, an identification confidence for each of the one or more recognized pathogen labels may be computed and added to the pathogen report. The outcome of this process may be a list of potential pathogens identified in the micrograph and may be accompanied by a confidence score for each prediction. In embodiments, features of the micrographs may be extracted as a vector representation of numerical values corresponding to a signature of the feature. The trained model may be used to recognize 160 the feature signature.

In embodiments, the trained pathogen model may include at least one of a residual network (ResNet) deep learning model, a few-shot learning (FSL) model, and a combination thereof.

In an exemplary embodiment, the model used is trained using Few-shot learning (FSL) machine learning, allowing the model to provide accuracy even when only a few samples of a pathogen are available for each of the possible reactant used. The trained model may include, for example, Siamese Network and Prototypical Networks trained using supervised learning, Meta-Learning Models such as Model-Agnostic Meta-Learning (MAML) and Reptile, trained using meta-learning techniques, Generative Models such as Generative Adversarial Networks (GANs), and Variational Autoencoders (VAEs), trained using unsupervised or semi-supervised learning techniques. Siamese Networks may learn to measure the similarity between two input micrographs. Prototypical Networks may learn a metric space where the distance between micrograph representations corresponds to their similarity, enabling classification with few examples per class. Meta-Learning Models, such as MAML or Reptile, may learn to adapt to new tasks or classes with limited labeled data.

Generative Models like GANs or VAEs may also be used for image generation or data augmentation, which may be beneficial in few-shot learning scenarios where the available labeled data is limited. Generative Models may, for example, generate additional data samples to supplement the training set.

Additionally, or alternatively, Attention Mechanisms may be incorporated into convolutional neural networks (CNNs) or other architectures to focus on relevant regions or features within a micrograph. This can improve performance in tasks such as object detection or micrograph classification, including few-shot learning settings.

As an alternative or complement to Few-Shot Learning (FSL), Residual Networks (or ResNets) deep learning models may be used. ResNets may be effective at addressing the vanishing gradient problem associated with training very deep neural networks, allowing ResNet models to learn complex and high-level features from micrographs without some of the training difficulties that may arise in networks of similar depth. ResNets may be particularly suited for tasks requiring the extraction and analysis of intricate patterns and features for pathogen identification 160, even when the network architecture is deep and the feature hierarchies are complex. ResNet architectures may efficiently learn hierarchical features from micrographs. In the initial layers, ResNet models may learn basic features such as edges and textures. As the information progresses through the network, deeper layers may learn more complex features that are specific to pathogens, such as shapes, patterns, and structures. Deep feature extraction capability may help identifying 160 and distinguishing between different types of pathogens. Additionally, the skip connections in ResNets may allow gradients to flow through the network directly, bypassing several layers at a time. This architecture may address the vanishing gradient problem by ensuring that the gradient can propagate back through the network without diminishing in strength. ResNets may be capable of learning highly abstract representations of the input data. ResNets may to generalize well to new, unseen micrographs of pathogens. Even if a particular pathogen strain has not been extensively represented in the training dataset, a well-trained ResNet model may accurately identify 160 the label by recognizing the general patterns and features that have been associated with that type of pathogen. ResNet models pre-trained on large datasets may be effectively adapted to the task of pathogen identification 160 through transfer learning. By finetuning the model on a smaller, task-specific dataset of micrographs, ResNets may leverage the generic features learned from the larger dataset and apply them to the specific task of identifying 160 pathogens. The architecture of ResNets may also allow for scalability in model depth. Various depths of ResNet models may be chosen (e.g., ResNet-50, ResNet-101, ResNet-152) depending on the complexity of the task and the available computational resources.

Few-Shot Learning (FSL) and Residual Networks (ResNets) may be combined to leverage the strengths of both approaches for pathogen identification 160. Integrating ResNets and FSL may enable hybrid models that first use ResNets for deep feature learning and then apply FSL techniques for classification based on these features. For example, in a combined FSL and ResNet approach, the ResNet architecture may be used as the backbone for feature extraction, where input micrographs may be processed to produce a rich representation or embedding of the visual content. The feature representations extracted by the ResNet may then be used in a Few-Shot Learning framework. For example, the extracted features may be used to compute class prototypes in the feature space. The FSL model may learn to classify new examples by comparing the feature representation-as processed by the ResNet-to the prototypes, even if only a few examples of each class are available. Meta-learning approaches, such as Model-Agnostic Meta-Learning (MAML) or Reptile, may also benefit from the deep and discriminative features extracted by ResNets. In this scenario, the ResNet may extract features that serve as the input to the meta-learning model, which may then learn how to quickly adapt to new tasks (e.g., identifying new or rare pathogens) with minimal data. Combining ResNets and FSL may also enable data augmentation and transfer learning strategies. Pre-trained ResNet models on large, diverse datasets may be finetuned with a small number of pathogen images in a few-shot setup, enabling the model to generalize better to new classes not seen during the initial training phase.

In embodiments, FSL and ResNets may be combined as part ensemble modeling, possibly with other modeling algorithms. Combining Few-Shot Learning (FSL) and Residual Networks (ResNets) as part of ensemble modeling, along with other algorithms may improve the performance and reliability of models used for pathogen identification 160. Ensemble modeling may use multiple learning algorithms or models simultaneously to obtain better predictive performance than could be obtained from any of the individual models alone. By including FSL and ResNets alongside other modeling algorithms, the ensemble may benefit from a broad spectrum of insights and approaches to the task, reducing the likelihood of systematic errors and improving overall accuracy. The predictions from each model in the ensemble may be combined through methods such as weighted voting or averaging, where the contribution of each model to the final prediction may be determined based on its performance or reliability. More sophisticated aggregation methods, such as stacking, may also be employed, where a meta-model is trained to optimally combine the predictions from the ensemble's base models. An ensemble that includes FSL and ResNets, along with other algorithms, may adapt to various challenges encountered in pathogen identification, such as changes in pathogen morphology or the introduction of new, rare pathogens. The ensemble may be designed to dynamically adjust the influence of each model based on the specific characteristics of the data being analyzed. As new data becomes available or as the task evolves, individual models within the ensemble may be retrained or replaced. By combining multiple models, ensemble methods may also help reduce the risk of overfitting, as the individual models' biases and variances may tend to cancel each other out, leading to more robust and generalizable predictions.

Persons skilled in the art will readily recognize that other deep learning techniques may be trained to classify the pathogens, each providing advantages over the other depending on the training dataset available.

In embodiments, the identification 160 of the pathogen may rely on multiple micrographs. For example, multiple frames of a video stream may provide motion used in the identification process 160. In other embodiments, bracketing may be used to provide different views of the sample under different parameters, and the set of micrographs may be used together to provide more accurate predictions. In other embodiments, the set of micrographs may comprise multiple angles of the sample such as a stereoscopic view of the pathogen, enabling depth analysis.

In embodiments, the one or more recognized pathogen labels may include at least one of a family, a name, a strain, a serotype, genetic information and a virulence factor. The family, name, strain, serotype, and virulence factor may be associated with different visual aspects of the pathogen and may sometimes be inferred from the visual representation of the pathogen. Different genetic information may be associated with similar visual representation but may be relevant in the pathogen identification. When multiple genetic variations of a pathogen may be associated with a common visual representation of the pathogen, multiple labels may be identified 160 for the same micrograph, each with the corresponding genetic information.

When a specific strain or serotype cannot be identified accurately for a pathogen, the identification 160 may fallback to only identifying the pathogen name without the strain or serotype. Furthermore, when strain or serotype identification 160 cannot be performed accurately, the one or more recognized pathogen labels may include one or more pathogen family labels. This may, for example, be an alternative outcome when the pathogen model has low confidence for specific pathogens, but high confidence on the family, allowing classification at a higher taxonomic level when lower-level classification is uncertain.

Once the pathogens have been identified 160, a report may be prepared 170 and presented back to the user describing the detected pathogens. In an exemplary embodiment, the report may include the probability and name of each pathogen identified (i.e., the pathogen labels).

In embodiments, scientific information about the one or more recognized pathogen labels may be searched within a scientific information repository. The scientific information about the one or more recognized pathogen labels may be added to the pathogen report. The report also provides decision trees to further improve the pathogen identification along with other information such as the identification protocol, types of diseases associated with the pathogen and treatment. Alongside the probability of each identified pathogen, confidence intervals or scores may be included to help users gauge the reliability of the identification. Micrograph images or visuals may highlight the detected pathogens within the sample and provide visual confirmation to help users understand the basis for the identification. A brief description of each identified pathogen, including its morphology, typical habitats, and common transmission modes, may also be provided within the report. If available, historical data or trends for the same or similar samples may be provided, in particular in situations where the pathogen is associated with an outburst within a geographical region. The report may also include general public health recommendations or guidelines for preventing the spread of diseases associated with the pathogens, including sanitation practices, quarantine measures, or vaccination recommendations. For each identified pathogen, the report may also provide a summary of known treatment options, including antibiotics, antiviral drugs, or other therapeutic measures. The report may also include references to relevant scientific literature or guidelines for further reading can help users seek more in-depth information or verify the recommendations provided in the report. In embodiments for monitoring specific pathogens or diseases, the report may comprise custom alerts for specific situations, such as the presence of potentially dangerous pathogens in milk or a parasite on a leaf. The report may include interactive features, such as clickable links to more detailed information, interactive decision trees, or options to explore data in more depth. The report may also provide strategies for the prevention and control of the identified pathogens, tailored to the specific environment or context (e.g., healthcare settings, agricultural practices, food industry standards).

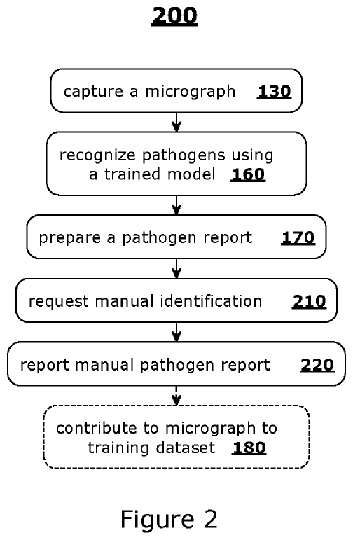

Reference is now made to the drawings in which FIG. 2 shows a flow chart depicting an exemplary method embodiment 200 for identifying pathogens from a sample in accordance with the teachings of the present invention.

In embodiments, a manual identification may be requested 210 of the micrograph. A manual pathogen report may be prepared 220 with one or more manual pathogen labels upon completing the manual identification. A network interface module 2170 may request 210 the manual identification of the micrograph and receive the manual identification of the micrograph. The decision may be guided by a desire from the user to increase the reliability of the detection when the automated result was unsatisfactory, or when the specific case justifies a manual inspection.

The micrograph may be manually inspected by a microbiologist or similarly trained professionals, and results may be manually entered and consolidated into a manual pathogen report 220. The manual input may be limited to the identification of the pathogen, and the report may be enriched therefrom to include other details presented hereinabove. The manual inspection may be performed after transmitting one or more micrographs using a network interface module 2170 to a remote review service. On the remote review service, micrographs may be enqueued for review. In embodiments, a priority may be associated with the review so that some reviews are executed in priority of others. Upon completion of the review, a notification may be issued to the pathogen identifying device 2100 to inform the user that the manual report is now available.

In embodiments, the micrograph and the one or more recognized pathogen labels may be contributed 180 to a training dataset. Contributing the micrographs reviewed manually may improve the quality of the training dataset, especially when the reviewed micrograph is considered to provide new insight or differs from the results from the trained pathogen model predictions. In other embodiments, micrographs captured by the user may be transmitted to a remote enrichment service, where the micrographs are reviewed and may be contributed to the training dataset even when predictions of the training model were accurate. Contributing well predicted micrographs may improve the quality of the training dataset by providing additional views of a given pathogen, improving predictions of a wider range of variations.

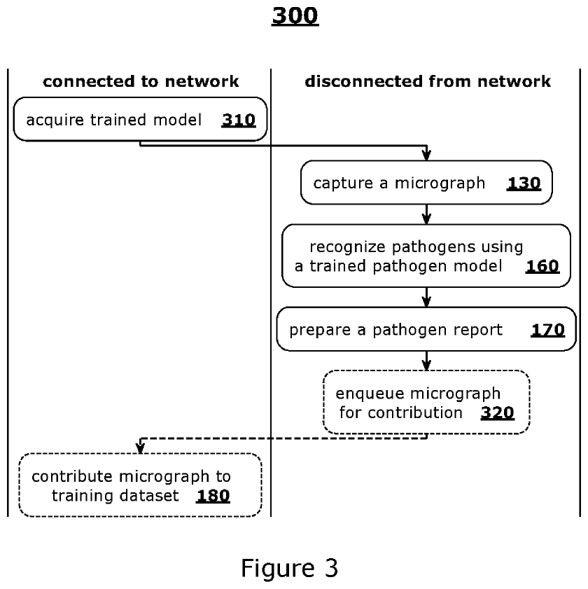

Reference is now made to the drawings in which FIG. 3 shows a flow chart depicting an exemplary method embodiment 300 for identifying pathogens from a sample in accordance with the teachings of the present invention.

In embodiments, the trained pathogen model may be acquired 310 while being connected to a remote pathogen model repository and the one or more pathogens may be recognized 160 while being disconnected from the remote pathogen model repository. In embodiments, the classifier module 2500 may be one of a local classifier module 2500A, a remote classifier module 2500B and a combination thereof.

When a reliable network, the micrographs or the video stream may be transferred to a remote classifier module 2500B. Using a remote classifier module 2500B may enable devices with limited processing capabilities to perform the pathogen identification 160. By keeping the remote classifier module 2500B in central locations, updates and improvements may be deployed without interfering with the users. In situations where a network connection to the remote classifier module 2500B may not be practical, a local classifier module 2500A may be collocated with the pathogen identifying device 2100. The local classifier module 2500A may be embedded within the pathogen identifying device 2100, or within the vicinity of the pathogen identifying device 2100, available through a reliable local connection. The trained pathogen model may be acquired 310 from a remote pathogen repository while a reliable network connection is available, or at a time when acquiring the model is non-critical. For the local classification module 2500A may attempt to retrieve an updated trained pathogen model over an unreliable connection while using an existing trained pathogen model without interfering with an ongoing identification 160 of pathogens. In embodiments, the identification 160 may be distributed between a local classifier module 2500A and a remote classifier module 2500B, for example, to leverage computing capabilities of the pathogen identifying device 2100 while maintaining some of the benefits of a centralized remote classifier module 2500B.

Initially, while connected to the internet or a network, the trained pathogen model may be downloaded or acquired from a remote repository. This repository may be a centralized location, typically hosted on a server, where models that have been developed, trained, and validated are stored. The acquisition of the model may involves transferring a copy of the model's parameters, architecture, and possibly other necessary components (like normalization parameters) to the local device. Once the model has been downloaded to the local device, the model may operate independently of the remote repository. With the model stored locally, users may input new micrograph data into the system, and the model may analyze the micrographs as inputs to recognize and identify pathogens. This process of inference or prediction may not require access to the remote repository, as all the computational processes involved in recognizing pathogens may be performed on the local device using the previously acquired model. Offline identification 160 may be particularly useful in remote locations where network infrastructure is sparse or non-existent. For instance, field researchers, healthcare workers in rural clinics, or scientists in remote laboratories may perform pathogen identification without the need for an internet connection.

In embodiments, the micrograph and the one or more recognized pathogen labels may be enqueued 320 for a micrograph contribution while being disconnected from a remote training dataset repository and the micrograph contribution to a training dataset may be contributed 180 upon reconnecting to the remote training dataset repository. While disconnected from the remote training dataset repository, pathogen identification 160 from micrographs may be performed. Data generated during the offline operations may be valuable for improving the trained pathogen model, including the micrographs themselves and the labels of pathogens recognized in these images. Enqueuing may include lining up the newly generated data (micrographs and associated pathogen labels) in a queue or a temporary storage area on the local device. Enqueuing is performed in preparation for future upload but may occur while the device is still offline. The data may be marked or stored in a way that indicates it is ready to be sent to the remote training dataset repository once connectivity is restored. Upon re-establishing a connection to the internet or the relevant network, the system may automatically begin the process of transferring the enqueued data from the local device to the remote training dataset repository. The addition of new micrographs and pathogen labels may improve the model's accuracy and robustness by providing it with more examples to learn from, especially if the new data includes novel or underrepresented pathogen instances. Valuable data may be collected in the field even without a network connection and contributes to the model's evolution and enhancement.

In embodiments, geolocation data may be associate with the micrograph (such as the location where the sample was taken) to monitor outbursts of pathogens in locations that would not be traditionally monitored due to lack of connectivity.

Reference is now made to the drawings in which FIG. 5 shows a logical modular representation of an exemplary system 2000 comprising a pathogen identifying device 2100. The pathogen identifying device 2100 comprises a memory module 2160, a processor module 2120, a capture module 2130 and a network interface module 2170. The pathogen identifying 2100 may also include a user interface module 2150.

The system 2000 may comprise a storage system 2300 for storing and accessing long-term (i.e., non-transitory) data and may further log data while the pathogen identifying 2100 is being used. FIG. 5 shows examples of the storage system 2300 as a distinct database system 2300A, a distinct module 2300 C of the pathogen identifying 2100 or a sub-module 2300B of the memory module 2160 of the pathogen identifying device 2100. The storage system 2300 may be distributed over different systems A, B, C. The storage system 2300 may comprise one or more logical or physical as well as local or remote hard disk drives (HDD) (or an array thereof). The storage system 2300 may further comprise a local or remote database made accessible to the pathogen identifying device 2100 by a standardized or proprietary interface or via the network interface module 2170.

The network interface module 2170 represents at least one physical interface that can be used to communicate with other network nodes. The network interface module 2170 may be made visible to the other modules of the pathogen identifying device 2100 through one or more logical interfaces. The actual stacks of protocols used by the physical network interface(s) and/or logical network interface(s) 2172-2178 of the network interface module 2170 do not affect the teachings of the present invention.

The processor module 2120 may represent a single processor with one or more processor cores or an array of processors, each comprising one or more processor cores. The memory module 2160 may comprise various types of memory (different standardized or kinds of Random Access Memory (RAM) modules, memory cards, Read-Only Memory (ROM) modules, programmable ROM, etc.).

A bus 2180 is depicted as an example of means for exchanging data between the different modules of the pathogen identifying device 2100. The teachings presented herein are not affected by the way the different modules exchange information. For instance, the memory module 2160 and the processor module 2120 could be connected by a parallel bus, but could also be connected by a serial connection or involve an intermediate module (not shown) without affecting the teachings of the present invention.

A capture module 2130 provides micrographs analyzed by the pathogen identifying device 2100 as described in more details hereinabove. More specifically, with reference being concurrently made to FIG. 1, and 2, the capture module 2130, the memory module 2160, the user interface module 2150, the classifier module 2500 and/or the processor module 2120 are selectively used to perform the steps 110 to 180. In some embodiments, the steps 160 and 170 may be executed on a remote classifier module 2500B with the micrograph and the resulting report being transmitted over the network 2200.

The variants of processor module 2120, memory module 2160 and network interface module 2170 usable in the context of the present invention will be readily apparent to persons skilled in the art. Likewise, even though explicit mentions of the capture module 2130, the memory module 2160, the user interface module 2150 and/or the processor module 2120 are not made throughout the description of the present examples, persons skilled in the art will readily recognize when such modules are used in conjunction with other modules of the pathogen identifying device 2100 to perform routine as well as innovative elements presented herein.

Various network links may be implicitly or explicitly used in the context of the present invention. While a link may be depicted as a wireless link, it could also be embodied as a wired link using a coaxial cable, an optical fiber, a category 5 cable, and the like. A wired or wireless access point (not shown) may be present on the link between. Likewise, any number of routers (not shown) may be present and part of the link, which may further pass through the internet.

The present invention is not affected by the way the different modules exchange information between them. For instance, the memory module and the processor module could be connected by a parallel bus, but could also be connected by a serial connection or involve an intermediate module (not shown) without affecting the teachings of the present invention.

A method is generally conceived to be a self-consistent sequence of steps leading to a desired result. These steps require physical manipulations of physical quantities. Usually, though not necessarily, these quantities take the form of electrical or magnetic/electromagnetic signals capable of being stored, transferred, combined, compared, and otherwise manipulated. It is convenient at times, principally for reasons of common usage, to refer to these signals as bits, values, parameters, items, elements, objects, symbols, characters, terms, numbers, or the like. It should be noted, however, that all of these terms and similar terms are to be associated with the appropriate physical quantities and are merely convenient labels applied to these quantities. The description of the present invention has been presented for purposes of illustration, but is not intended to be exhaustive or limited to the disclosed embodiments. Many modifications and variations will be apparent to those of ordinary skill in the art. The embodiments were chosen to explain the principles of the invention and its practical applications and to enable others of ordinary skill in the art to understand the invention in order to implement various embodiments with various modifications as might be suited to other contemplated uses.

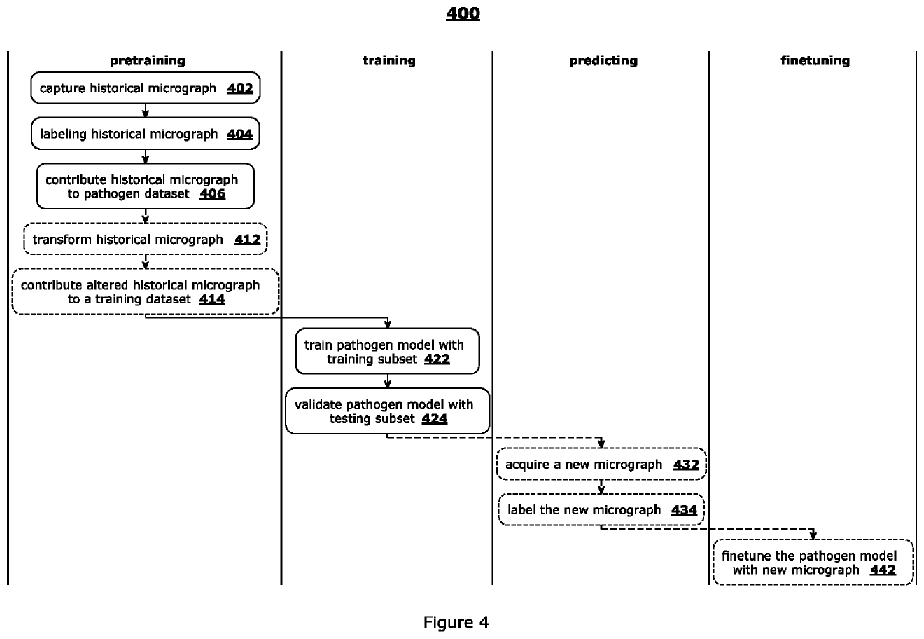

A second aspect of the teachings presented herein relates to devices, systems, computer programs and methods for training a pathogen model is provided. Reference is now made to the drawings in which FIG. 4 shows a flow chart depicting an exemplary method embodiment 400 for training a pathogen model in accordance with the teachings of the present invention.

The method includes capturing 402 one or more historical micrographs of a historical sample, applying 404 a historical pathogen label on the one or more historical micrographs into one or more labeled historical micrographs and contributing 406 the one or more labeled historical micrographs to a pathogen dataset.

The capturing 402 may be broader than the meaning of capturing 130 previously described hereinabove. Capturing 402 may include, for example, obtaining micrographs from existing databases, or obtaining images of synthetic micrographs generated from a model. The term historical micrographs and historical sample may refer to micrographs and samples that are not necessarily being captured in real-time or within a short period of time preceding the training. The historical micrographs and historical samples may have been acquired before the training process and may be reused for multiple executions of the training process.

The historical pathogen label being applied 404 on the one or more historical micrographs (thereby obtaining a labeled historical micrograph) may be identified by trained microbiologist or experts with the required knowledge of microbial morphology to identify the pathogens on the historical micrographs. In some cases, visual inspection may not be sufficient and additional laboratory tests may be performed on the samples to accurately identify the pathogen on the micrograph. For example, Polymerase Chain Reaction (PCR), culture methods, Enzyme-linked Immunosorbent Assay (ELISA), Mass Spectrometry, Fluorescent in-situ Hybridization (FISH), sequencing, or antimicrobial susceptibility testing may be performed to accurately identify the pathogens.

The label may include the name of the pathogen. Additionally, the label may include the strain or serotype, genetic information, the virulence factors, and the method of identification. The strain or serotype may allow the model to distinguish pathogens that may exhibit unique morphological features or patterns that are distinguishable in micrographs. Inclusion of genetic markers or specific sequences may help differentiate between pathogens that are visually similar under microscopy but genetically distinct, allowing the model to report the uncertainty between multiple similar-looking pathogens with different genetic markers. In addition, some pathogens may carry specific virulence factors that may influence their morphology or growth patterns observable in micrographs. Including virulence factors in the labels may enable the model to recognize pathogen variants with unique pathogenic properties. The method used for the initial identification (e.g., PCR, sequencing) may provide context about the reliability of the pathogen label and any specific features that were used to confirm its presence, which might also be visible in micrographs. The conditions under which the micrograph was captured (e.g., magnification, lens properties such as the aperture, illumination properties, shutter speed, use of polarization filters and properties thereof), may also be relevant when the model is used to predict similarly captured micrographs.

The labeled historical micrographs may then be contributed 406 to a pathogen dataset. Ideally, the pathogen dataset includes a wide variety of micrograph representing each of the pathogen to be recognized.

In embodiments, the one or more labeled historical micrographs may be transformed 412 into one or more altered historical micrographs and the one or more altered historical micrographs may be contributed 414 to a training dataset. Transforming original images to create altered historical micrographs may enhance the robustness and generalization capability of machine learning models in contexts like pathogen recognition from micrographs. The transformations may introduce variability into the training dataset that helps the model learn to recognize pathogens under different conditions and from varied perspectives, thus improving its accuracy and reliability when applied to new, unseen images. The altered micrographs may also palliate for a limited number of samples for rare pathogens.

The one or more labeled historical micrographs may be transformed 412 using at least one of a blurring, a scaling, a rotation, a skewing, and a noise addition. Images may be rotated by varying degrees to help recognize pathogens regardless of their orientation in the micrograph. For instance, rotating an image of bacteria by 90, 180, and 270 degrees may simulate different orientations that bacteria might appear in real samples. Images may be scaled up or down (zoomed in or out) to simulate differences in magnification. Scaling may ensure that the model can identify pathogens even when they appear smaller or larger than in the training images. For example, scaling an image to 90% or 110% of its original size may mimic slight variations in microscope settings. Cropping parts of images may simulate the effect of pathogens being partially out of the field of view or focus. Cropping may train the model to recognize pathogens even when only a portion is visible. Cropping may be random or follow specific patterns to enhance variability. Varying the brightness and contrast may prepare the model for variations in lighting and staining quality. For instance, increasing or decreasing the brightness by a certain percentage may simulate different lighting conditions encountered during sample preparation. Adding random noise to images may simulate imperfections in the imaging process, such as graininess due to high ISO settings or dust on the camera sensor. Noise may include Gaussian noise, salt-and-pepper noise, or other types of image noise. Images may be flipped horizontally or vertically. Flipping may help the model learn that the orientation of the sample does not change its fundamental characteristics. Flipping images may mirror the appearance of the pathogen, adding another layer of variability. The color channels (e.g., hue, saturation) may be slightly altered to simulate variations in color that might result from different staining techniques or microscope camera settings. Applying skewing or elastic deformations may simulate the natural variability in shape that pathogens might exhibit due to the preparation of the slides or the inherent flexibility of biological tissue. Skewing, or elastic deformations may help the model learn to recognize pathogens even when shapes are not perfectly consistent with the majority of training images.

The method further includes training 422 the pathogen model using supervised reinforcement learning on a training subset of the pathogen dataset and validating 424 the pathogen model using a testing subset of the pathogen dataset.

Initially, the model may be trained on a subset of the pathogen dataset using supervised learning techniques. Training may involve providing the model with labeled micrographs (input) and their corresponding pathogen labels (output), allowing the model to learn the associations between the visual features of the micrographs and the pathogen types. Techniques from FSL may be particularly useful here if the dataset has limited examples of certain pathogens. ResNets may serve as a feature extractor in this phase, learning complex patterns and structures from the data. After an initial supervised learning phase, reinforcement learning techniques may be used to further refine the model. Unlike the traditional reinforcement learning setup where an agent interacts with an environment, in supervised reinforcement learning for pathogen identification, the model may receive feedback (rewards or penalties) based on the model's performance on specific tasks, such as correctly or incorrectly identifying pathogens. This feedback loop may allow the model to adjust its parameters to improve performance iteratively. The model may receive positive feedback when it correctly identifies a pathogen, encouraging it to reinforce the patterns or features associated with that correct identification. Conversely, incorrect identification may result in negative feedback, prompting the model to adjust its approach to reduce similar errors in the future.

The model may then be validated using a distinct subset of the pathogen dataset not seen during the training phase. This testing subset may serve as a proxy for real-world data, allowing the evaluation of the model's generalization capabilities and its ability to accurately identify pathogens in new, unseen micrographs. During validation, various performance metrics such as accuracy, precision, recall, and F1 score may be calculated to assess the model's effectiveness in pathogen identification.

When embodied as a system or device. One or more processors may capture 402 one or more historical micrographs of a historical sample, apply 404 a historical pathogen label on the one or more historical micrographs into one or more labeled historical micrographs and contribute 406 the one or more labeled historical micrographs to a pathogen dataset. The pathogen dataset may be stored on a storage module capable of persisting and retrieving a large amount of data. The storage module may be collocated with the one or more processors or accessible over a network connection. The one or more processors may further train 422 the pathogen model using supervised reinforcement learning on a training subset of the pathogen dataset and validate 424 the pathogen model using a testing subset of the pathogen dataset.

In embodiments, one or more new micrographs may be acquired 432 of a new sample, the one or more new micrographs may be labeled 434 into one or more labeled new micrographs. The pathogen model may be finetuned 442 using the one or more labeled new micrographs into a finetuned pathogen model. New micrographs may represent data that may contain previously unseen variations of pathogens, new pathogens, or variations in imaging conditions. Continuous acquisition of new micrographs may enable the model to improve based on the most current and varied representations of pathogens. The new micrographs may be as described hereinabove, often by trained microbiologists or using automated systems with expert validation. The labeling process may involve identifying and annotating the pathogens present in the micrographs. The labels may provide a ground truth needed to teach the model about the characteristics of different pathogens as observed in the new micrographs. Once a new micrograph has been labeled, it may be added to the pathogen training dataset. In order to finetune the model, a specific finetuning dataset may also be used, containing micrographs that were not in the original pathogen training dataset. The model is then finetuned using the newly acquired and labeled micrographs. Finetuning may involve making incremental adjustments to the model's parameters, allowing it to learn from the new data without forgetting the knowledge it has already acquired. Finetuning may be done in several ways, such as adjusting the learning rate to ensure that the model's existing knowledge is preserved while integrating new information, or by employing techniques like transfer learning where the model is initially trained on a large dataset and then refined with a smaller, specific dataset. After finetuning, the model's accuracy and robustness may have been improved by incorporating the new data. Finetuning is generally a faster and less resource-intensive process than training, and may allow more frequent updates to the pathogen model. Finetuning may enable the pathogen model to adapt to any changes in pathogen appearance, emergence of new pathogen strains, or variations in image capture conditions that may not have been present in the original training dataset.

1-17. (canceled)

18. A method for training a pathogen model, the method comprising:
capturing (402) one or more historical micrographs of a historical sample;
applying (404) a historical pathogen label on the one or more historical micrographs into one or more labeled historical micrographs;
contributing (406) the one or more labeled historical micrographs to a pathogen dataset;
training (422) the pathogen model using supervised reinforcement learning on a training subset of the pathogen dataset; and
validating (424) the pathogen model using a testing subset of the pathogen dataset.

19. The method of claim 18, further comprising:
transforming (412) the one or more labeled historical micrographs into one or more altered historical micrographs; and
contributing (414) the one or more altered historical micrographs to a training dataset.

20. The method of claim 19, wherein the one or more labeled historical micrographs is transformed (412) using at least one of a blurring, a scaling, a rotation, a skewing, and a noise addition.

21. The method of claim 18, further comprising:
acquiring (432) one or more new micrographs of a new sample;
labeling (434) the one or more new micrographs into one or more labeled new micrographs; and
finetuning (442) the pathogen model using the one or more labeled new micrographs into a finetuned pathogen model.

22-39. (canceled)

40. A system for training a pathogen model comprising:
one or more processors configured to:
capture (402) one or more historical micrographs of a historical sample;
apply (404) a historical pathogen label on the one or more historical micrographs into one or more labeled historical micrographs;
contribute (406) the one or more labeled historical micrographs to a pathogen dataset;
train (422) the pathogen model using supervised reinforcement learning on a training subset of the pathogen dataset; and
validate (424) the pathogen model using a testing subset of the pathogen dataset.

41. The system of claim 40, wherein:
the one or more processors are further configured to:
transform (412) the one or more labeled historical micrographs into one or more altered historical micrographs; and
contribute (414) the one or more altered historical micrographs to a training dataset.

42. The system of claim 41, wherein:
the one or more processors are further configured to:
transform (412) the one or more labeled historical micrographs, using at least one of a blurring, a scaling, a rotation, a skewing, and a noise addition.

43. The system of claim 42, wherein:
the one or more processors are further configured to:
acquire (432) one or more new micrographs of a new sample;
label (434) the one or more new micrographs into one or more labeled new micrographs; and
finetuning (442) the pathogen model using the one or more labeled new micrographs into a finetuned pathogen model.

44-60. (canceled)

61. A device for training a pathogen model comprising:
one or more processors configured to:
capture (402) one or more historical micrographs of a historical sample;
apply (404) a historical pathogen label on the one or more historical micrographs into one or more labeled historical micrographs;
contribute (406) the one or more labeled historical micrographs to a pathogen dataset;
train (422) the pathogen model using supervised reinforcement learning on a training subset of the pathogen dataset; and
validate (424) the pathogen model using a testing subset of the pathogen dataset.

62. The device of claim 61, wherein:
the one or more processors are further configured to:
transform (412) the one or more labeled historical micrographs into one or more altered historical micrographs; and
contribute (414) the one or more altered historical micrographs to a training dataset.

63. The device of claim 62, wherein:
the one or more processors are further configured to:
transform (412) the one or more labeled historical micrographs, using at least one of a blurring, a scaling, a rotation, a skewing, and a noise addition.

64. The device of claim 63, wherein:
the one or more processors are further configured to:
acquire (432) one or more new micrographs of a new sample;
label (434) the one or more new micrographs into one or more labeled new micrographs; and
finetuning (442) the pathogen model using the one or more labeled new micrographs into a finetuned pathogen model.

65-81. (canceled)

82. A non-transitory computer-readable medium storing a set of instructions for training a pathogen model, the set of instructions comprising:
one or more instructions that, when executed by one or more processors of a device, cause the device to:
capture (402) one or more historical micrographs of a historical sample;
apply (404) a historical pathogen label on the one or more historical micrographs into one or more labeled historical micrographs;
contribute (406) the one or more labeled historical micrographs to a pathogen dataset;
train (422) the pathogen model using supervised reinforcement learning on a training subset of the pathogen dataset; and
validate (424) the pathogen model using a testing subset of the pathogen dataset.

83. The non-transitory computer-readable medium of claim 82, wherein the one or more instructions further cause the device to:
transform (412) the one or more labeled historical micrographs into one or more altered historical micrographs; and
contribute (414) the one or more altered historical micrographs to a training dataset.

84. The non-transitory computer-readable medium of claim 83, wherein the one or more labeled historical micrographs is transformed (412) using at least one of a blurring, a scaling, a rotation, a skewing, and a noise addition.

85. The non-transitory computer-readable medium of claim 82, wherein the one or more instructions further cause the device to:
acquire (432) one or more new micrographs of a new sample;
label (434) the one or more new micrographs into one or more labeled new micrographs; and
finetune (442) the pathogen model using the one or more labeled new micrographs into a finetuned pathogen model.
